(19) United States
(12) Patent Application Publication
(10) Pub. No.: US 2024/0319130 A1
(43) Pub. Date: Sep. 26, 2024

(54) AN ELECTRODE SYSTEM BASED ON DIFFERENTIAL OXIDANT RESPONSE FOR THE DETECTION OF FREE CHLORINE

(71) Applicant: INDIAN INSTITUTE OF TECHNOLOGY MADRAS (IIT MADRAS), Chennai (IN)

(72) Inventors: Pradeep THALAPPIL, Chennai (IN); Chaudhari KAMALESH, Chennai (IN); Servida TULLIO, Bangalore (IN); V VISHNU, Chennai (IN); B K Spoorthi, Chennai (IN)

(21) Appl. No.: 18/579,377

(22) PCT Filed: Jul. 14, 2022
(86) PCT No.: PCT/IN2022/050637

§ 371 Date: Jan. 15, 2024

(30) Foreign Application Priority Data
Jul. 15, 2021 (IN) 202141031970

(51) Int. Cl. G01N 27/30; G01N 33/18

(52) U.S. Cl. CPC G01N 27/301; G01N 27/302; G01N 33/18

(57) ABSTRACT

The present invention relates to an electrode system for detecting residual chlorine in water. Active electrode of the device was prepared by a very simple method in which the antimony pellet was prepared using high pressure and sintering at higher temperatures. An advanced version of the same was prepared by electrochemical coating of antimony on copper rods. A combination of thin platinum wire and antimony coated electrode was used to test residual chlorine in water. The said device responds to the changes in residual chlorine concentrations in water in the range of 0-4 ppm. To increase the accuracy of the electrode system two platinum-antimony electrodes were used by adding an inline cartridge of oxidant absorptive material. This method was used for the sensing of pH using the same electrode system and further pH compensation of chlorine signal.

### FIELD OF THE INVENTION

The present invention relates to an electrode system for the detection of free chlorine in water. More specifically, the present invention relates to a platinum, antimony and activated carbon (or similar oxidant absorptive material)-based electrode system for the detection and quantification of residual chlorine in water.

### BACKGROUND OF THE INVENTION

Antimony electrode was introduced over 100 years ago for pH measurement. The electrode potential is governed by equations, Sb→Sb³⁺+3e and Sb₂O₃+6H⁺→2Sb³⁺+3H₂O; K=[Sb³⁺]/[H⁺]³. Obviously, the concentration of H⁺ affects the equilibrium constant and therefore the electrode potential. Although this appears simple, there are a large number of chemical reactions which can affect the potential generated. In the case of chlorine containing solutions, equilibria of the kind, Cl₂(aq)+2H₂O→HOCl+Cl⁻+H₃O⁺ and H₂O+HOCl→ClO⁻+H₃O⁺ are important, which are affected by pH.

Chlorine is an important disinfectant used for all the public utilities. The concentration of free chlorine in water is important to be monitored as too less of it is not enough for disinfection but too much of it produces undesired halogenated compounds such as trihalomethanes. A residual chlorine concentration of 2-3 mg/L is considered as optimum, while in India, it is kept below 2 mg/L. Measurement of free chlorine at affordable cost if performed at every home, safety of water supplied can be guaranteed.

Chlorine in water hydrolyses as per the equation, Cl₂+2H₂O→2HOCl+2H⁺+2e. A part of the chlorine is consumed for oxidising residual organic matter in the water and only the remaining is available for disinfection. HOCl is the principal species available for disinfection, which itself dissociates to form ClO⁻ and H⁺. A solution of free chlorine therefore will have HClO and ClO⁻ as the two species important for disinfection, whose concentrations are roughly equal at pH 7.5 and 25° C. At pH lower than 7.5, HOCl dominates and at pH above 7.5, ClO⁻ dominates, this is the case in the pH window of 5 to 9. Additional complication arises from the disproportionation reaction, 2HClO+ClO⁻→ClO₃⁻+2Cl⁻+2H⁺. In the case of drinking water, the pH range of relevance is narrow, from 6.5 to 8.5.

HClO and ClO⁻ are considered free chlorine as one mole of chlorine gas produces one mole of either of these species. The concentration of these species depends upon pH of the solution. It is important to mention that other species such as hypochloroniumacidium ion (H₂OCl⁺) and chloronium (or chlorinium) ion (Cl⁺) have also been reported, which are all oxidizing species.

The foregoing suggests that the free chlorine in water is extremely sensitive to the pH and in other words, pH is a measure of free chlorine, especially within a narrow window of concentrations of relevance to disinfection. This is possible as pH is often regulated at the water treatment plant and change in chlorine can happen in the process of distribution of the treated water. If chlorine and pH are measured at the source, change in chlorine at the service point is measurable with an antimony electrode. This is important as for each of the source; there can be often thousands of homes using the treated water. Such a chlorine measurement is particularly useful as this would be an affordable alternative with an antimony price of $7 per kg.

The present invention relates to the development of a new electrode system for the inline measurement of free chlorine in water, affordably.

### EXISTING TECHNOLOGIES IN THE ART

Chlorine (Cl₂), hypochlorite (ClO⁻) and hypochlorous acid (HClO) have been used extensively as disinfectants due to their strong oxidizing properties [Dong Y, et al., Analytical Chemistry, 84(19), 8378-8382.]. Chlorine is in use from long time for the disinfection of drinking water, wastewater and swimming pool water. All the chlorine mentioned above altogether is called as free residual chlorine. However, level of free chlorine in water needs to be controlled strictly as at higher levels it is harmful for human beings and animals. Chlorine concentration of 4 mg/L is known as a maximum residual disinfectant level (MRDL) [Xu J, et al., Sensors and Actuators B: Chemical, 156(2), 812-819]. Higher concentrations of free chlorine in water may affect immune system, cardiovascular system, respiratory tract and it can also lead to cancer [Dong Y, et al., Analytical Chemistry, 84(19), 8378-8382.]. Multitude of sensing schemes have been designed and reported for the sensing of free chlorine in water. These methods include colorimetry[Piraud C, et al., Analytical Chemistry, 64(6), 651-655], amperometric probes[Xu J, et al., Sensors and Actuators B: Chemical, 156(2), 812-819; U.S. Pat. No. 7,790,006 B2; CN103185742 A], nanoparticle based methods [Dong Y, et al., Analytical Chemistry, 84(19), 8378-8382.], chemiluminescence[Belz. M et al., Sensors and Actuators B: Chemical, 39(1-3), 380-385], and optical methods [Belz M et al., Sensors and Actuators B: Chemical, 39(1-3), 380-385]. DPD (diethyl-p-phenylenediamine) based titrimetry is one of the most popular and reliable methods for the determination of free chlorine in water. Such colorimetric methods are suitable for lab based accurate determination of ions in water. Amperometric methods have been developed extensively for the detection of free chlorine [U.S. Pat. No. 7,790,006 B2; CN103185742 A]. Amperometry can be used either to detect end point of the titrations or for the direct detection of chlorine in water. Briefly a combination of inert material electrode and a copper counter electrode have been used for the measurements but this technique was found to be unreliable due to the dependence of measurements on the mass-transfer coefficient at the electrode surface by the particular hydrodynamics prevailing in the measuring cell [Piraud C, et al., Analytical Chemistry, 64(6), 651-655]. The major issue with the amperometric sensors is the kinetics of chlorine reduction, which may become irreversible for contaminated electrode surfaces. Use of graphite electrode based chronoamperometric chlorine sensing has been also reported[WO2017020133 A1]. Surface passivated Graphene quantum dots (GQDs) with fluorescent properties have been also reported for the sensing of chlorine based on fluorescence quenching [Dong Y, et al., Analytical Chemistry, 84(19), 8378-8382.]. Monochromatic (290 nm) absorption based measurements of the hypochlorite ion (ClO⁻) have been developed in the past. But such optical measurements have been found to be varying with the water samples and it was necessary to calibrate each time with different types of water samples [Aoki T et al., Anal. Chem., 1983, 55, 209-212]. It was also dependent on the reagents which are required to keep optics clean and safe from fouling. Based on a similar approach, a fiber optics based system has been developed recently for chlorine monitoring.

Various other free chlorine or pH sensing technologies have been patented in the past. These technologies are based on different principles as explained in the following paragraph. Among the reported methods, one of the most popular technologies is the use of chlorine permeable membrane. Use of chlorine permeable membrane with working and auxiliary electrodes fabricated using precious metals such as gold/platinum makes it an efficient chlorine sensor [KR200344894 Y1]. Another patented technology is based on the use of metal silicide containing materials for the detection of free chlorine [WO2020248542 A1]. Another patent reports the use of a three electrode system based on platinum disc electrodes (as an actuating and auxiliary electrode) with saturated calomel electrode as a reference for the sensing of free chlorine [KR20040009344 A]. Some of these systems make use of antimony for the construction of electrode. One of the systems with antimony working electrode and ceramic reference electrode has been reported for the sensing of pH [U.S. Pat. No. 5,497,091 A].

Various strategies have been used for the construction of antimony electrodes. Typically, well-polished antimony has been used for enhanced sensitivity. Incorporation of antimony in polymer substrate was also reported for the construction of pH probe [WO2000067010 A1]. A system based on a combination of a sensor electrode (noble metals, antimony or bismuth) and reference electrode made of oxides/hydroxides of zinc or magnesium was reported [U.S. Pat. No. 6,653,842 B2]. This system has been demonstrated for the sensing of pH and ORP [U.S. Pat. No. 6,653,842 B2].

However, each of these techniques have pros and cons such as use of toxic chemical reagents, instability of long term operation, low detection sensitivity, poor selectivity, etc. As a result, reliable chlorine sensing electrodes using antimony are difficult. Upon long term operation of the electrodes, contamination of electrodes may lead to instability and drift in the measurements.

In the present invention, we address each of these limitations by stable coating of antimony on copper surface. The coated antimony surface was treated with sulfur containing moieties to improve the stability.

### OBJECTS OF THE INVENTION

An object of the present invention relates to an electrode system for detecting free chlorine in water.

Another object of the present invention relates to a platinum-antimony electrode system for the detection and quantification of residual chlorine in water.

An object of the present invention relates to electrode system fabricated with a layer of Antimony modified with Oxygen and Sulfur. Here Antimony can be coated on Copper or similar conducting substrate.

Yet another object of the present invention relates to an electrode cell which uses an inline cartridge of oxidant absorptive material between the two antimony-platinum (Sb—Pt) electrode systems. Here, the first Sb—Pt system can be used to quantify chlorine in water and the second Sb—Pt system can be used to detect the changes in pH. Once the exact pH is known from the later Sb—Pt system, the signal from prior SB—Pt electrode can be corrected for the pH dependent changes in chlorine signal.

### SUMMARY OF THE INVENTION

The present invention relates to an electrode system for detecting free chlorine in water, namely a platinum-antimony electrode system for the detection and quantification of residual chlorine in water.

In one embodiment, the present invention relates to a platinum-antimony electrode system for the inline measurement of free chlorine in water, where the concentration of free chlorine in tap water stocks was verified using DPD (diethyl-p-phenylenediamine) based titrimetric method.

In another embodiment, the present invention relates to an electrode cell which uses an inline cartridge or a barrier of oxidant absorptive material between the two antimony-platinum (Sb—Pt) electrode systems. The inline cartridge/barrier uses activated carbon (AC) as the oxidant absorptive material. Here prior Sb—Pt system (before inline cartridge) can be used to quantify chlorine in water and later Sb—Pt can be used to sense pH as well as correct the Chlorine signal by pH compensation.

The Sb—Pt electrodes before AC act as a sensing system here to sense the oxidants like chlorine in water. The antimony (Sb) electrodes exhibit a relatively higher selectivity towards HOCl as compared to ClO⁻. Once the response due to oxidants like chlorine is known, the signal from Sb—Pt electrodes after AC is used as a reference to detect the changing pH.

The direct measurement of free chlorine with pH compensation based on the differential oxidant response of the same electrode system (Sb—Pt) is a unique method proposed in this invention.

Other aspects of the invention will be set forth in the description which follows, and in part will be apparent from the description, or may be learnt by the practice of the invention

### BRIEF DESCRIPTION OF THE DRAWINGS

FIG. 1A shows the line diagram of platinum-antimony electrode representing platinum wire (1), antimony pellet (2), and 3D printed body (3). The pellet is made from a 13 mm antimony disk of 5 mm thickness and a platinum wire of 0.5 mm dia. Electrode body was printed with ABS (Acrylonitrile butadiene styrene). B shows the line diagram of platinum-Sb@Cu electrode representing platinum wire (4), Sb@Cu (5), and 3D printed body (6). Electrode body was printed with ABS (Acrylonitrile butadiene styrene).

FIG. 2A depicts the variations in the electrode cell potential, between an antimony pellet/platinum wire cell, as a function of residual chlorine in water. B depicts the variations in the electrode cell potential, between antimony coated copper rods/platinum wire cell, as a function of residual chlorine in water.

FIG. 3 shows the variations in the electrode cell potential, as a function of time when it was exposed to the flow of varying free chlorine concentrations in water.

FIG. 4 illustrates the line diagram of a free chlorine sensor with pH compensation. It is comprised of two Sb@Cu—Pt and Pt based electrode cells with inline activated carbon cartridge.

FIG. 5A shows the oxidant response of Sb@Cu—Pt at different pH. B shows the pH response of Sb@Cu—Pt at different oxidant concentrations. In the absence of oxidant only pH response can be obtained for the pH compensation of prior signal.

FIG. 6 shows stability of Sb@Cu—Pt response with time. It can be seen that the electrode response stabilizes after ˜10 days and stays constant for over two months.

FIG. 7 Photograph of the Sb@Cu—Pt electrode showing antimony coated Cu rod element and platinum wire.

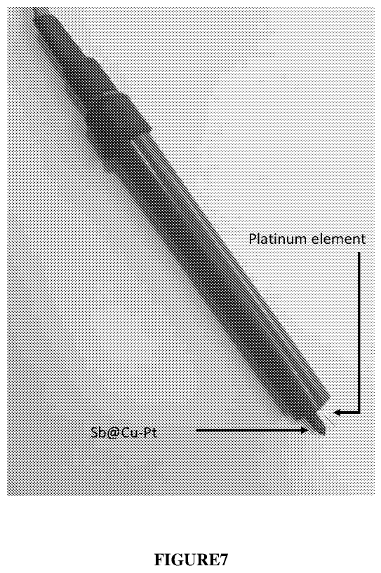

Referring to the drawings, the embodiments of the present invention are further described. The figures are not necessarily drawn to scale, and in some instances the drawings have been exaggerated or simplified for illustrative purposes only. One of ordinary skill in the art may appreciate the many possible applications and variations of the present invention based on the following examples of possible embodiments of the present invention.

### DETAILED DESCRIPTION OF THE INVENTION

The following description is presented to enable any person skilled in the art to make and use the embodiments, and is provided in the context of a particular application and its requirements. Various modifications to the disclosed embodiments will be readily apparent to those skilled in the art, and the general principles defined herein may be applied to other embodiments and applications without departing from the scope of the present invention. Thus, the present invention is not limited to the embodiments shown, but is to be accorded the widest scope consistent with the principles and features disclosed herein.

The present invention relates to an electrode system for detecting free chlorine in water, specifically to a platinum-antimony electrode system for detecting and quantifying of residual chlorine in water, where the concentration of free chlorine in water stocks was verified using DPD (diethyl-p-phenylenediamine) based titrimetric method.

In another embodiment, the present invention relates to an electrode cell which uses an inline cartridge or a barrier of oxidant absorptive material between the two antimony-platinum (Sb—Pt) electrode systems. The inline cartridge/barrier uses activated carbon (AC) as the oxidant absorptive material. Here prior Sb—Pt system (before inline cartridge) can be used to quantify chlorine in water and later Sb—Pt can be used to sense pH as well as correct the chlorine signal by pH compensation.

The Sb—Pt electrodes before AC act as a sensing system here to sense the oxidants like chlorine in water. The antimony (Sb) electrodes exhibits a relatively higher selectivity towards HOCl as compared to ClO⁻. Once the response due to oxidants like chlorine is known, the signal from Sb—Pt electrodes after AC is used as a reference to detect the changing pH.

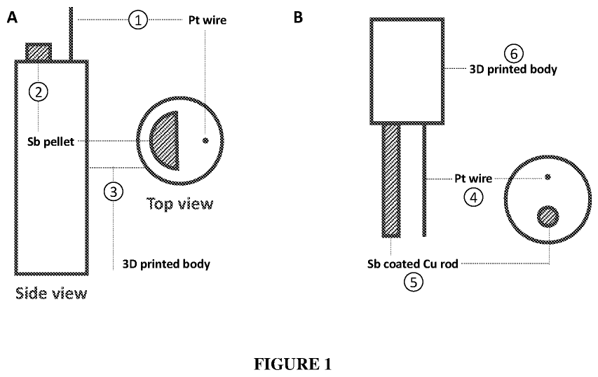

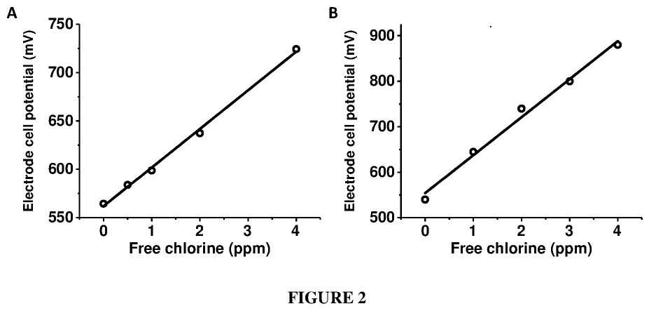

An electrode cell comprising of an inert platinum electrode and antimony counter electrode was fabricated for these measurements. Platinum wire of 500 micron diameter was used with the antimony pellet. The antimony pellet was prepared using a load of 10 ton for 20 sec and sintering at higher temperatures (550 C). The antimony pellet and the platinum wire were fixed with a 3D printed stick like structure as shown in FIG. 1. Stick was printed using ABS (Acrylonitrile butadiene styrene) as a 3D printing material. This set-up was tested using standard pH meter in mV mode. The data obtained for the measurements of free chlorine in tap water is shown in FIG. 2. Free chlorine concentration water was adjusted using 200 ppm sodium hypochlorite stock solution. The concentration of free chlorine in tap water was verified using DPD (diethyl-p-phenylenediamine)-based titrimetric method. It was found that response of electrode was linear with respect to the changes in free chlorine concentration.

An advanced version of electrode cell comprising of platinum electrode and antimony coated copper rod (Sb@Cu) was fabricated for these measurements. Platinum wire of 500 micron diameter was used with the antimony pellet. The antimony was coated on copper rods of 70 mm length and 3 mm in diameter by an electrochemical approach. Coating was done in two steps. In the first step platinum counter electrode was used. Electrolyte was prepared by dissolving pure antimony metal in 50% HNO₃ solution. The precipitated antimony oxide in the prior process was dissolved in a 50 mL solution comprising of 9 M NaOH and 1.3 M Na₂S. This solution was used as an electrolyte for antimony coating on Cu rods at 10 V and 8.2 mA/cm². After this, Sb@Cu rods were washed thoroughly with deionized water (DI). In the next step, Sb@Cu was modified with oxygen and sulfur. These modified Sb@Cu rods were used for the next measurements and validation studies.

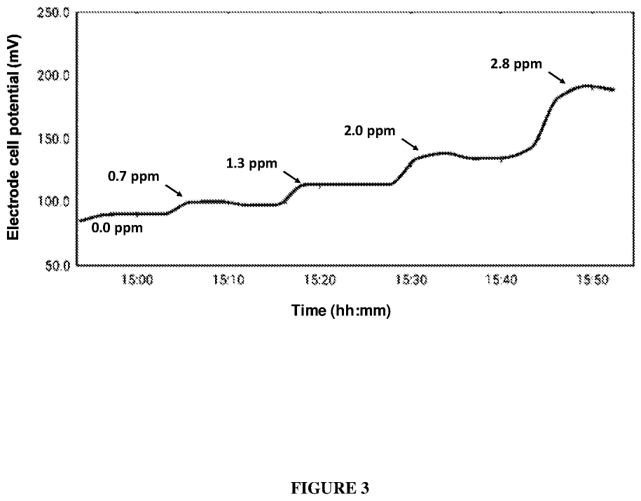

After the verifications using steady state measurements, inline measurements were conducted. For inline measurements, sensor was connected with a test skid where dosimeter was used for an adjustment of free chlorine concentration through controlled dosing of sodium hypochlorite stock solution. The results obtained are shown in the FIG. 3. The sensor was found to be responsive to changes in free chlorine concentration while used for inline measurements.

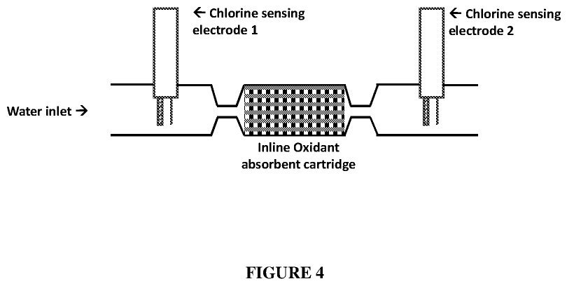

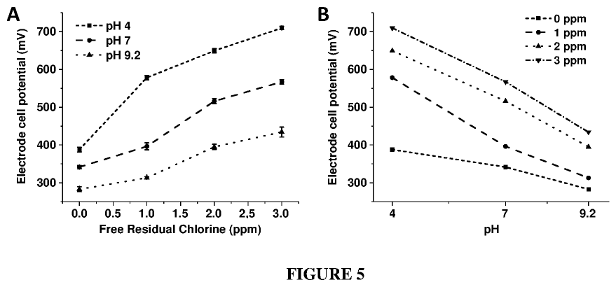

The schematic of the chlorine sensor with pH compensation is shown in FIG. 4 where the signal generated by an aqueous solution under test is measured in the presence and absence of oxidant (here free residual chlorine) using two different Sb@Cu—Pt electrode cells. The potential generated on the first electrode can be corrected by the potential generated on the second electrode which responds only to the changes in pH. The pH sensing ability of the Sb@Cu—Pt was tested at three different pH. FIG. 5A shows changes in the response of Sb@Cu—Pt at different pH. Suggesting requirement of pH compensation. The same electrode response has been plotted to show (FIG. 5B) linear response of Sb@Cu—Pt to varying pH at the same oxidant concentration. When these changes are used to correct the prior data, we can get an absolute change in signal only due to chlorine concentration. The uniqueness of the system lies in the construction which facilitates use of stable Sb@Cu—Pt electrodes in differential oxidant environment. The differential oxidant response helps in the pH compensation using two units of the same electrode system.

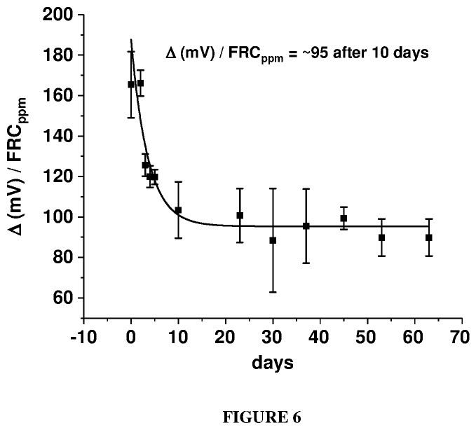

Due to oxidant based sensing activity of these electrode systems, the sensor can also measure other oxidants like ozone. As most of the disinfecting agents are effective oxidants, this electrode sensing system can be used to measure effectiveness of disinfecting agents. The stability of the Sb@Cu—Pt responsiveness to chlorine was studied for two months (FIG. 6). It was found that the system response stabilizes after a pre-treatment of ˜10 days and remains constant over a period of two months.

It may be appreciated by those skilled in the art that the drawings, examples and detailed description herein are to be regarded in an illustrative rather than a restrictive manner.

We claim:

1. An electrode system for detecting free chlorine in water, the said system comprises:
i. an electrode cell comprised of antimony coated copper rods and a platinum wire (Sb@Cu—Pt);
ii. two Sb@Cu—Pt electrode cells separated by oxidant absorptive material;
characterized in that, the said first Sb@Cu—Pt electrode cell when dipped in an aqueous solution produces a potential that varies as a function of the oxidizing chlorine species in solution to detect the free chlorine in water, and the second Sb@Cu—Pt perform measurements in the absence of oxidant for the detection of the changes in pH.

2. The electrode system as claimed in claimed 1, wherein the signal from second Sb@Cu—Pt is used for the pH compensation of chlorine signal.

3. The electrode system as claimed in claimed 1, wherein the said electrode system detects free chlorine in the range of 0.1 to 4 ppm, in water.

4. The electrode system as claimed in claim 1, wherein oxidizing species includes HOCl and ClO⁻.

5. The electrode system as claimed in claim 1, wherein the second Sb@Cu—Pt electrode cell is used as a reference to detect the changing pH.

6. The electrode system as claimed in claim 1, wherein the oxidant absorptive layer is activated carbon or a material with similar properties.

7. The electrode system as claimed in claim 1, wherein the antimony is coated on copper or any other conducting substrate.
